SEQUENCE LISTING (12 sequences)

SEQ ID NO: 1 - DNA, 4232 bp, Plasmodium falciparum
atggataggttgaacaagtatttgtctgttcttttttttttcttctttttcttttttcttaatatctttcaaataaaaactgaggaaaagcataagtcacttagcaaagataacgcttatactttatattttaaaaatgtcacaaaaaaatgattaatacaaataaaagtagaaatatatatatatatatatatagtcatttatttatttatactttttatatatttcctcaaattgtagagacaaaaatatgtagttcgtaggagcgaaggttctccatacagaaattttcttttgtatgttgaggaaatttatcaagttactgtggtgtaccctattaaatatctttttgttaataaataaattatataaaataatatatgcataaataaatataaatataaataaatatatatatatatatatatatatatatattattgtaaccattcaataggatattcctctaaaatattatgaatatttcaagtcctataaaaagaaagatatagataataaaaatttaaaacaattttgtaatataattaaagagcaacatgaaataaataatgaacaacaaaaatgtttctgttgttattgtcgtcaagatgatatggtatttccattattttctcattttaattataaaaaagcaaagtttaaatgtaacaagaaaggtgacaataaatatatatgcgttaacatttcttatacttggctgtatttatattgttatccatataatcaagcattaataatatcaatatatatatatatatatatatatatgtgtgtgtatatttatttatttattttttttttttttagaacaagaagaggatatagttctgaccttgtcttgtttgaaaaagaaagacaaaatttttcattcttttttcatacaacaaaattacaacttttttatgttaaatgttacattacaacaatttggtattgtgttcatataagatttatacaaactgtcttattatataatatgatatatttattttatattttttttttttttttttttttttctttctcttagatttgaatgacgaatatttcataaacataaatgtaatataaacaaaaaaaaaaaaaaaaatatatatatatatatatatataggtctgtattaatattttcgtttatataaaaatatacaggataagtctactctagaaagaaaaacttatctccttgattcttcaaattatgaaatagatgacggtatatatataaagaaaaaaaaaaaaaaaaaatacacaaacacacatatatatatatatatatatatatatatattatttctctatatttgaggaaaataagaaatgattaattattattattattattactacaaattgtataattcaaaaaatgaatatacatacgatatacctgcttttctcctttttgcagaagtatttaatgtaaagataaaatttaatgtccaaaaaaattacagagtaaataataaaaaactatacatgcatttttgctattactccaatatatatatatatatatataaatctacatttttaaattagaatgacgatattgagggcaactacctttttgtggactcaaatatttttaaggatagggttaaataaaaatataatattaatttatatatatatttagtaacaaacccaaaaaaaaaaaaaaaaaaaaaaaaaatatggttatatgtaaatattttcctttgtgttaatattattacagataatattagaagactcatttaaaggtaagcctttaattttttaataaaataaaaaaagaaaaacatgtaacatatatatatatatatatatttatttatttatttatttatttatttgtggttatgtgaatttgttatattatcctatggttcattcaaaatatatattaatttatttaaacattcatatttgaaagatggagatattttaaaaaattcagttattgtaaaatctcatcatgttgatgaacatggacttacatgtaacaaaataattggttatccgatttattggaatagaggaaaaggattgtgtcgattaaaaagaaatgaatgttttcataaacaggtttctacatttttaaatttagaaaagaaattaagctcgaaaaataatatgagctataattattctttctttatgataagaaagttacatatggatgaatataatgagatagatgaaggttttgaaaaaacgaatgaatatatatataatataaataaacttaataaaataaatgaaataatagatgattataagtataataatacggatagatataaatatgatattaagattgataataatgagaacttattagaatatataaaaaataattattatattggtttacaaaaagaaagacatcattttatagatataaaatcacataatgaagaaatattaaatatatcatatgacgaggaaggtataggagagtccgtatcctttgtacatatattaagtaattgttacaattataataatgaaaaaaaaaatatctcatgtagtatttatataagtatatggaatgaagaagatatagagaaagaaataaatgtttttataaaatgtaaacaaaggattgttaatgatataacacaactaaataaaaaattatttttatgtaaaaagtgtgaagaaactattcttataaaatttgaacctatatataatatgttgctagcatcatgtcatgttttaataacaaaagaggaaagaaatcttgatataaaaaatatatatgaagatcctaatattaggggtgcaaattataaagagcaacattttctttttgttaaagatgaagaaaatgtagaggcatcgcataattataaatataaaaaaggaaaatataaaacttatgattatgatgatgatatggtaaatatggatcaaagagtacaaaataaagaatatcataagatagataaaatatataataataataataataataataatgataatatataccacaaaaatgtatacattattaaagaagtacctcttaaattcgatgaacaattctataggccaaacgatcataacataatgttattatataatacaatgaactttgttgaaaatgtagaagattatattttaagatataaaaggaaattctttttttttataaacaaatcagggaatattgtaaaaataattttattcttattcttagttattatattatgtatttttataataccaagtttacctttctgtacatatttttttattaaattaaaatggtttcataaactaaaaacaattcgtctattgatattatggaaagtacaagatacattcacattttttaaaaagatacccaggaaagtatggagattcataaaaaaaattgcaaaaaaatgtaaacgggtgctttttaaaatatttataaaatataaaaaggatcaagaacttattgatcaagaatataaagaaaaattacaaagaaaaagaaaaaaagaaagaaaaaaaaaacaaatcagaaaaaaatatcaacttcaaaaaaaggagaaggaaattttgagaaaaaaaagatataaaaagttgattgaaaatgaaaaaatatttcaaaaggaaatgaaaaaagaattaatgcataaaagaaataataaaataaaacacgaaaacaaacatgaagaaaattattatataaacaacaaaaatgatcatacatatggtcataaaagtaaaacgcagaaacattataaaaaatatcatgataaatatgaagataaatatcatcataaatatcataagaaagaaccaaaacaaaaaaatatatataatcatttaaataatcctcataatccatcatataacgatttagaaaaaaatcaatcattaacttcaaacacaaattcttctcaagaatattatgaaaagaaaaaatatgaaaatactctttcatctgctgaacatataaatataaaaaacactcatcataaaagtaaattaaatacttttaataataaaaacacatatgataaccaatatattaaaaaagaaccatatgataaggataatatatattctgatttctatgctataaaaaacaatgaccaacaaagtattctccaagaaaaaaagaaaaatagaagaagcatatccaacatatcaaaaatttataaaaataatgataccatacaactatctaataaggatagtaaaactagtagaaaagatcttttaccataa

SEQ ID NO: 2 - protein, 999 aa, Plasmodium falciparum
MDRLNKYLSRQKYVVRRSEGSPYRNFLLYVEEIYQVTVVYPIKYLFDIPLKYYEYFKSYKKKDIDNKNLKQFCNIIKEQHEINNEQQKCFCCYCRQDDMVFPLFSHFNYKKAKFKCNKKEQEEDIVLTLSCLKKKDKIFHSFFIQQNYNFFMLNVTLQQFDLNDEYFININDKSTLERKTYLLDSSNYEIDDEVFNVKIKFNVQKNYRNDDIEGNYLFVDSNIFKDRIILEDSFKDGDILKNSVIVKSHHVDEHGLTCNKIIGYPIYWNRGKGLCRLKRNECFHKQVSTFLNLEKKLSSKNNMSYNYSFFMIRKLHMDEYNEIDEGFEKTNEYIYNINKLNKINEIIDDYKYNNTDRYKYDIKIDNNENLLEYIKNNYYIGLQKERHHFIDIKSHNEEILNISYDEEGIGESVSFVHILSNCYNYNNEKKNISCSIYISIWNEEDIEKEINVFIKCKQRIVNDITQLNKKLFLCKKCEETILIKFEPIYNMLLASCHVLITKEERNLDIKNIYEDPNIRGANYKEQHFLFVKDEENVEASHNYKYKKGKYKTYDYDDDMVNMDQRVQNKEYHKIDKIYNNNNNNNNDNIYHKNVYIIKEVPLKFDEQFYRPNDHNIMLLYNTMNFVENVEDYILRYKRKFFFFINKSGNIVKIILFLFLVIILCIFIIPSLPFCTYFFIKLKWFHKLKTIRLLILWKVQDTFTFFKKIPRKVWRFIKKIAKKCKRVLFKIFIKYKKDQELIDQEYKEKLQRKRKKERKKKQIRKKYQLQKKEKEILRKKRYKKLIENEKIFQKEMKKELMHKRNNKIKHENKHEENYYINNKNDHTYGHKSKTQKHYKKYHDKYEDKYHHKYHKKEPKQKNIYNHLNNPHNPSYNDLEKNQSLTSNTNSSQEYYEKKKYENTLSSAEHINIKNTHHKSKLNTFNNKNTYDNQYIKKEPYDKDNIYSDFYAIKNNDQQSILQEKKKNRRSISNISKIYKNNDTIQLSNKDSKTSRKDLLP

SEQ ID NO: 3 - protein, 22 aa, Plasmodium falciparum
RQDDMVFPLFSHFNYKKAKFKC

SEQ ID NO: 4 - protein, 32 aa, Plasmodium falciparum
RQDDMVFPLFSHFNYKKAKFKCNKKEQEEDIV

SEQ ID NO: 5 - protein, 46 aa, Plasmodium falciparum
FCCYCRQDDMVFPLFSHFNYKKAKFKCNKKEQEEDIVLTLSCLKKK

SEQ ID NO: 6 - protein, 45 aa, Plasmodium berghei
FCCYCTQSSIFPLYFQLKYRRKKLLCNKKDNNEDVILTFSCLKKK

SEQ ID NO: 7 - protein, 45 aa, Plasmodium yoelii
FCCYCTQSSIFPLYSKLKYRRKKLLCNKKDNNENVILTLSCLKKK

SEQ ID NO: 8 - protein, 47 aa, Plasmodium vivax
FCCHCSPHPVFPLYSSLKYRRQKVLCRRRVTDGEEAVVLTLKCLKKS

SEQ ID NO: 9 - protein, 46 aa, Plasmodium falciparum
ESCICPSYHLFKNDNSIKRAKLKCTYFNMLFTDSAIVYSRHCAIMD

SEQ ID NO: 10 - protein, 48 aa, Toxoplasma gondii
RCCWCSEVLAFTHINNMKRGNFRCNWFAPPRALELVTETLYDQCEAGK

SEQ ID NO: 11 - protein, 52 aa, Chlamydomonas reinhardtii
FCCECSSSQVWDDTFGSSKERTRANLDCDFWSDPLDILIGRKPVSAHCLTFD

SEQ ID NO: 12 - protein, 43 aa, Plasmodium falciparum
CCYCRQDDMVFPLFSHFNYKKAKFKCNKKEQEEDIVLTLSCLK

(19) United States
(12) Patent Application Publication
(10) Pub. No.: US 2023/0390372 A1
(43) Pub. Date: Dec. 7, 2023

(54) COMPOSITIONS AND METHODS FOR TARGETING PLASMODIUM MALE GAMETE PROTEIN TO BLOCK MALARIA PARASITE TRANSMISSION

(71) Applicant: SEATTLE CHILDREN'S HOSPITAL D/B/A SEATTLE CHILDREN'S RESEARCH INSTITUTE, Seattle, WA (US)

(72) Inventors: Sudhir Kumar, Seattle, WA (US); Stefan H.I. Kappe, Seattle, WA (US); Ashley M. Vaughan, Seattle, WA (US); David Noah Sather, Seattle, WA (US)

(73) Assignee: SEATTLE CHILDREN'S HOSPITAL D/B/A SEATTLE CHILDREN'S RESEARCH INSTITUTE, Seattle, WA (US)

(21) Appl. No.: 18/043,497

(22) PCT Filed: Aug. 27, 2021
(86) PCT No.: PCT/US2021/048047

§ 371 Date: Feb. 28, 2023

Related U.S. Application Data
(60) Provisional application No. 63/071,854, filed on Aug. 28, 2020.

(51) Int. Cl. A61K 39/015; C07K 14/445; C07K 16/20

(52) U.S. Cl. CPC A61K 39/015; C07K 14/445; C07K 16/205

(57) ABSTRACT

The disclosure provides immunogenic peptides comprising at least a portion of a Plasmodium HAP2 paralog (“HAP2p”) protein, immunogenic compositions comprising or encoding the immunogenic peptides, antibodies binding the immunogenic peptides, and methods of preventing Plasmodium transmission incorporating the peptides, compositions, and/or antibodies. In some embodiments, the immunogenic peptide has a sequence comprising a sequence with at least 80% identity to a sequence of at least 10 continuous amino acids of SEQ ID NO:2, a Plasmodium HAP2 paralog (“HAP2p”) protein.

### CROSS-REFERENCE TO RELATED APPLICATION

This application claims the benefit of U.S. Provisional Application No. 63/071,854, filed Aug. 28, 2020, the entire disclosure of which is incorporated herein by reference in its entirety.

### STATEMENT OF GOVERNMENT LICENSE RIGHTS

This invention was made with Government support under AI127338 awarded by the National Institutes of Health (NIH). The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3399-P29WO_Seq_List_FINAL_20210825_ST25.txt. The text file is 19 KB; was created on Aug. 25, 2021; and is being submitted via EFS-Web with the filing of the specification.

### BACKGROUND

Malaria has a tremendous impact on human health, killing hundreds of thousands annually and creating a major impediment for social and economic development of nations in malaria-endemic areas, particularly in sub-Saharan Africa. Parasites of the genus Plasmodium, the causative agents of malaria, are transmitted to the vertebrate host through the saliva of an infected Anopheles mosquito. After transmission, Plasmodium parasites, in the sporozoite stage, travel quickly through the blood stream to the liver. Sporozoites that infect hepatocytes grow and replicate within the infected hepatocyte, producing tens of thousands of blood stage-infectious merozoites. Merozoites infect red blood cells, where they undergo further development and replication, after which they cause the rupture of the red blood cell, releasing a new wave of merozoites into the blood. Most of the new merozoites continue to repeat the replicative cycle through more red blood cells. This cycling of infection and rupturing of the red blood cells manifests in the potentially severe symptoms associated with malaria, such as fever, chills, weakness, malaise, and enlarged spleen.

A minority of the cycling merozoites eventually develop into male or female gametocytes that remain in circulation within the body until being taken up in a blood meal by a new mosquito. Assuming the new mosquito is compatible (i.e., another Anopheles mosquito), the gametocytes proceed to develop into gametes and fuse to form a diploid zygote. Zygotes develop into motile ookinete forms, which penetrate the wall of the mosquito's midgut and form oocysts. The oocyst undergoes numerous rounds of division to eventually produce infective sporozoites that can be injected into the next vertebrate host, thus repeating the lifecycle.

Various efforts have been made to prevent infection and/or transmission of malaria, including use of anti-mosquito insecticides, practical uses of bed netting, anti-malarial pharmaceuticals, and vaccines that stimulate immune responses against the human stage parasites to lower the parasitic burden and symptoms of disease. However, transmission of malaria continues despite these efforts.

Accordingly, despite the advances in understanding of Plasmodium biology and factors affecting transmission, there remains a need for additional approaches to block the transmission of the parasite from infected individuals. This disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides an immunogen comprising:
an immunogenic fragment of a Plasmodium HAP2 paralog (HAP2p) protein; and
an immunogenic carrier operatively coupled to the peptide.

In some embodiments, the HAP2p protein is a Plasmodium falciparum HAP2p protein.

In another aspect, the disclosure provides an immunogen comprising:
a peptide comprising a sequence with at least 80% sequence identity to at least 10 consecutive amino acids of a sequence corresponding to SEQ ID NO:4; and
an immunogenic carrier operatively coupled to the peptide.

In some embodiments, the peptide comprises a sequence with at least 85%, at least 90%, or at least 95% to at least 10 consecutive amino acids of a sequence corresponding to SEQ ID NO:4. In some embodiments, the peptide comprises a sequence with at least 80%, at least 85%, at least 90%, or at least 95% to at least 15 or at least 20 consecutive amino acids of a sequence corresponding to amino acids 61-116 of SEQ ID NO:2. In some embodiments, the peptide comprises a sequence with at least 80% sequence identity to at least 10 consecutive amino acids of a sequence corresponding to SEQ ID NO:3. In some embodiments, the peptide comprises a sequence with at least 85%, at least 90%, or at least 95% to at least 10 consecutive amino acids of a sequence corresponding to SEQ ID NO:3. In some embodiments, the peptide comprises a sequence with at least 80%, at least 85%, at least 90%, or at least 95% to at least 15 or at least 20 consecutive amino acids a sequence corresponding to SEQ ID NO:4. In some embodiments, the peptide comprises or consists essentially of a sequence corresponding to SEQ ID NO:4.

In some embodiments, the immunogenic carrier is or comprises keyhole limpet hemocyanin, Concholepas concholepas hemocyanin, bovine serum albumin (BSA), cationized BSA, ovalbumin, and the like.

In another aspect, the disclosure provides an immunogenic composition comprising the immunogen as described herein, and an adjuvant. In some embodiments, the adjuvant is or comprises one of incomplete Freund's adjuvant, aluminum phosphate, aluminum hydroxide, alum, high molecular weight polysaccharides, glycoproteins, microparticles, liposomes, and the like.

In another aspect, the disclosure provides a vector comprising a nucleic acid sequence encoding a peptide comprising a sequence with at least 80% sequence identity to at least 10 consecutive amino acids of a sequence corresponding to SEQ ID NO:4.

In some embodiments, the peptide comprises a sequence with at least 80%, at least 85%, at least 90%, or at least 95% to at least 15 or at least 20 consecutive amino acids of a sequence corresponding to SEQ ID NO:4. In some embodiments, the peptide comprises a sequence with at least 80%, at least 85%, at least 90%, or at least 95% to at least 10, at least 15, or at least 20 consecutive amino acids of a sequence corresponding to amino acids 95-116 of SEQ ID NO:2. In some embodiments, the peptide comprises or consists essentially of a sequence corresponding to SEQ ID NO:3. In some embodiments, the nucleic acid is operatively linked to a functional promoter.

In another aspect, the disclosure provides a cell comprising the vector disclosed herein, or a progeny thereof.

In another aspect, the disclosure provides a method of preventing or inhibiting transmission of a Plasmodium from an infected or susceptible host. The method comprises administering to the host an effective amount of the immunogen as described herein, an effective amount of the immunogenic composition as described herein, or an effective amount of the vector as described herein. In some embodiments, the method further comprises administering the effective amount two or more times to the host. In some embodiments, the Plasmodium is Plasmodium falciparum and the susceptible host is a human.

In another aspect, the disclosure provides an antibody or antigen binding derivative thereof that specifically binds to an immunogenic fragment of a Plasmodium HAP2 paralog (HAP2p) protein. In some embodiments, the HAP2p protein is a Plasmodium falciparum HAP2p protein. In some embodiments, the HAP2p fragment comprises a peptide comprising a sequence with at least 80% sequence identity to at least consecutive amino acids of a sequence corresponding to SEQ ID NO:4. In some embodiments, the HAP2p fragment comprises a peptide comprising a sequence with at least 80%, at least 85%, at least 90%, or at least 95% to at least 15 or at least 20 consecutive amino acids of a sequence corresponding to SEQ ID NO:4. In some embodiments, the HAP2p fragment comprises a peptide comprising a sequence with at least 80%, at least 85%, at least 90%, or at least 95% to at least 10, at least 15, or at least 20 sequence identity of a sequence corresponding to SEQ ID NO:3. In some embodiments, the peptide comprises or consists essentially of a sequence corresponding to SEQ ID NO:3. In some embodiments, the antibody or antigen binding derivative thereof is chimeric or humanized.

In another aspect, the disclosure provides a method of preventing or inhibiting transmission of a Plasmodium from susceptible host, comprising administering to the host an effective amount of the antibody or antigen binding derivative thereof as described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a sequence alignment of protein fragment sequences of the HAP2p protein from various Plasmodium species encompassing the Cd loop domain (i.e., P. falciparum, SEQ ID NO:5; P. berghei, SEQ ID NO:6; P. yoelii, SEQ ID NO:7; and P. vivax, SEQ ID NO:8). The alignment also includes the paralogous fragment from Plasmodium falciparum HAP2 protein (SEQ ID NO:9). The alignment also includes the protein fragment sequence from the HAP2 of Toxoplasma gondii (SEQ ID NO:10) and Chlamydomonas reinhardtii (SEQ ID NO:11). The Cd loop domain is indicated by the line bar and associated box. Similar residues in vertical columns of the alignment are indicated in bold.

FIG. 2 is a sequence alignment of protein fragment sequences of the HAP2p protein from various P. falciparum strains encompassing the Cd loop domain demonstrating that the sequence is 100% conserved and is set forth herein as SEQ ID NO:12. The Cd loop domain is indicated.

FIG. 3 graphically illustrates the number of oocysts observed in the midgut of mosquitos taking a blood meal spiked with control (PBS or preimmune blood) or antibodies to the PfHAP2p Cd loop. The graph shows that presence of antibodies that selectively bind the Cd loop domain block fertilization and, thus, prevent progression of the parasitic lifecycle in the mosquito host. The different between each control group and the “immune” experiment (i.e., with a presence of antibodies) was statistically significant at p<0.001. “n” denotes numbers of mosquitoes dissected and “ug/ml” denotes amount of antibody used.

FIG. 4 graphically illustrates the number of oocysts observed in the midgut of mosquitos taking a blood meal with control or antibodies to the PfHAP2p Cd loop from a second experiment. The graph is consistent with results illustrated in FIG. 3, namely that presence of antibodies that selectively bind the Cd loop domain block fertilization and, thus, prevent progression of the parasitic lifecycle in the mosquito host. The different between each control group and the “immune” experiment (i.e., with a presence of antibodies) was statistically significant at p<0.001. “n” denotes numbers of mosquitoes dissected and “ug/ml” denotes amount of antibody used.

FIGS. 5A-5C illustrate the confirmation of a locus knockout. FIG. 5A schematically illustrates the primer design to detect the successful knockout of the P. falciparum HAP2p gene. FIG. 5B is a table indicating the expected size of amplified band depending on the knockout status and primer used. FIG. 5C is an image of a gel confirming the knockout of the HAP2p locus.

FIGS. 6A and 6B graphically illustrate that the P. falciparum genetic knockout parasites do not transmit to mosquitos where sexual reproduction would normally occur. FIG. 6A graphically illustrates the exflagellation centers observed in fields of view. FIG. 6B graphically illustrate the oocysts observed in the midgut of mosquitos after a blood meal. These results indicate that, while exflagellation proceeds normally in HAP2p genetic knockout parasites, the gametocytes do not result in fertilization or oocyst development in the mosquito midgut.

FIGS. 7A-7F illustrate the expression of HAP2p in P. falciparum male gametocytes. FIGS. 7A, 7B, and 7C are a series of photomicrographs showing the results of immunofluorescent staining to characterize the HAP2p protein in developing gametocytes. FIG. 7A specifically illustrates the localization of HAP2p in gametocyte stages II-V. FIG. 7B illustrates the localization of HAP2p in activated gametocytes including emerging male gametocytes. FIG. 7C illustrates the localization of HAP2p in stage V gametocyte, activated gametocytes and free male gamete (microgamete) in transgenic PfHAP2p-mCherry parasite line. FIGS. 7D, 7E, and 7F schematically illustrate the primer design to detect the successful PfHAP2p gene tagging with fluorescent reporter mCherry.

FIG. 8 is a series of micrographs showing binding of monoclonal antibody E5 with stage V gametocytes. Anti-PfHAP2p antibody is used as a positive control.

### DETAILED DESCRIPTION

The present disclosure is based on the inventors' discovery of a HAP2 paralog (referred to herein as “HAP2p”) in Plasmodium spp. that is expressed on male gametocytes and is necessary for gamete fusion to produce diploid oocyst in the mosquito host. As described in more detail below, the inventors demonstrated that antibodies generated against the Cd loop of the HAP2p protein can be ingested by mosquitoes in a blood meal that contains Plasmodium gametes. When ingested in this manner, the anti-HAP2p antibodies block the fusion of the male and female gametes, thereby preventing progression of the lifecycle in the mosquito host. This discovery has significant implications for control of Plasmodium transmission from infected humans and, therefore, can be incorporated into malaria control strategies.

In accordance with the foregoing, in one aspect the present disclosure provides an immunogen comprising: an immunogenic fragment of a Plasmodium HAP2 paralog (HAP2p) protein; and an immunogenic carrier operatively coupled to the peptide.

As used herein, the terms “Plasmodium” or “parasite” refer to any parasite that belongs to the genus Plasmodium. In some embodiments, the Plasmodium organism can infect human hosts, such as, for example, P. falciparum, P. vivax, P. ovale, P. malariae, and P. knowlesi. In some embodiments, the Plasmodium organism is P. falciparum. In some embodiments, the Plasmodium organism is P. vivax or P. ovale. In other embodiments, the Plasmodium organism can infect other vertebrate hosts, such as non-human primates and rodents. Examples of such Plasmodium organisms include P. yoelii, P. berghei, P. chabaudi, P. vinckei, and P. cynomolgi.

An exemplary HAP2p protein is from P. falciparum and is set forth herein as SEQ ID NO:2. In one embodiment, the immunogenic fragment contains part or all of the Cd loop. An exemplary Cd loop from P. falciparum is at positions 95-126 of SEQ ID NO:2, and is reflected in the sequence set forth herein as SEQ ID NO:4. The immunogenic fragments encompassed by this aspect can contain at least 5, 6, 7, 8, 9, 10, 11, 12, 13, 14, 16, 17, 18, 19, 20, 21, 22, 23, 24, 25, 26, 27, 28, 29, 30, or 31 consecutive amino acids, or all 32 amino acids of the Cd loop (such as set for in SEQ ID NO:4). Description of additional embodiments of the immunogenic fragment is provided below.

In some embodiments, the HAP2p protein, or fragment thereof is a Plasmodium falciparum HAP2p protein or fragment thereof.

In another aspect, the disclosure provides an immunogen comprising: an immunogenic peptide comprising a sequence with at least about 80%, about 85%, about 90%, about 95%, or about 98% sequence identity to at least 10 consecutive amino acids of a sequence of SEQ ID NO:2. For example, in some embodiments, the immunogen comprises a peptide comprising a sequence with at least about 80%, about 85%, about 90%, about 95%, or about 98% sequence identity (e.g., including 100% identity) to at least 10, at least 11, at least 12, lease 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50, consecutive amino acids of a sequence of SEQ ID NO:2. In some embodiments, the immunogen further comprises an immunogenic carrier operatively coupled to the peptide.

In some embodiments, the peptide comprises a sequence with at least about 80%, about 85%, about 90%, about 95%, or about 98% sequence identity to at least 10 consecutive amino acids of a sequence corresponding to amino acids 95-126 of SEQ ID NO:2 (i.e., corresponding to the sequence set forth in SEQ ID NO:4). For example, in some embodiments, the immunogen comprises a peptide comprising a sequence with at least about 80%, about 85%, about 90%, about 95%, or about 98% sequence identity (e.g., including 100% identity) to fhap2f. In some embodiments, the peptide comprises or consists essentially of a sequence of SEQ ID NO:4. In some embodiments, the peptide comprises a sequence corresponding to at least a portion of the Cd loop of a Plasmodium HAP2p protein, an illustrative example of which is a Plasmodium falciparum HAP2p protein Cd loop sequence as set forth in SEQ ID NO:4.

In some embodiments, the peptide comprises a sequence with at least about 80%, about 85%, about 90%, about 95%, or about 98% sequence identity (e.g., including 100% identity) to at least 10 consecutive amino acids of the sequence set forth in SEQ ID NO:3. In some embodiments, the immunogen comprises a peptide comprising a sequence with at least about 80%, about 85%, about 90%, about 95%, or about 98% sequence identity to at least 10, at least 11, at least 12, lease 13, at least 14, at least 15, at least 16, at least 17, at least 18, at least 19, at least 20, at least 21 consecutive amino acids, or all the amino acids of the sequence set forth in SEQ ID NO:3.

In some embodiments, the immunogenic peptide comprises a sequence with at least a portion of a portion of the Cd loop of a Plasmodium HAP2p protein plus additional sequence of the HAP2p protein. For example, the peptide can comprise a sequence with at least about 80%, about 85%, about 90%, about 95%, or about 98% sequence identity to the sequence set forth in one of SEQ ID NOS:5-8, or a fragment with at least 10, at least 11, at least 12, lease 13, at least 14, at least 15, at least 16, at least 17, at least 18, at least 19, at least 20, at least 21, at least 22, at least 23, at least 24, at least at least 26, at least 27, at least 28, at least 29, at least 30, at least 31, at least 32, at least 33, at least 34, at least 35, at least 36, at least 37, at least 38, at least 39, at least 40 amino acids thereof.

In exemplary embodiments, the immunogenic peptide comprises a sequence as set forth in one of SEQ ID NOS:3-8 and 12.

In some embodiments, the immunogenic peptide has a sequence that is distinct from a sequence reflecting a wild-type HAP2p fragment.

As indicated, the various aspects of the disclosure can incorporate an immunogenic carrier operatively linked to the peptide (e.g., the immunogenic fragment of the Plasmodium HAP2 paralog (HAP2p) protein).

The term “operatively linked” refers to a structural linkage, covalent or non-covalent, that retains the peptide (e.g., the immunogenic fragment of a Plasmodium HAP2 paralog (HAP2p) protein) and immunogenic carrier in close proximity such that the carrier facilitates an immune response to the immunogen. Various technologies for linking immunogenic peptides to carrier proteins are known and are encompassed by this disclosure.

The disclosure encompasses any immunogenic carrier that facilitates generation of an immune response, e.g., antibody production, against the coupled peptide. Persons of ordinary skill in the art can readily identify and incorporate any immunogenic carrier appropriate for this purpose. Many appropriate immunogenic carriers are known and available in the art, such as keyhole limpet hemocyanin, Concholepas concholepas hemocyanin, bovine serum albumin (BSA), cationized BSA, ovalbumin, and the like.

In another aspect, the disclosure provides an immunogenic composition. The immunogenic composition comprises an immunogen as described above, and an adjuvant.

Adjuvants that facilitate induction of a strong immune response in antibody-producing animals or known in the art and are encompassed by the disclosure. Exemplary adjuvants encompassed by the present disclosure is include or comprise incomplete Freund's adjuvant, aluminum phosphate, aluminum hydroxide, alum, high molecular weight polysaccharides, glycoproteins, microparticles, liposomes, and the like.

In another aspect, the disclosure provides a vector. The vector of the disclosure comprises a nucleic acid sequence encoding a peptide comprising a sequence as described herein above.

A “vector” is a nucleic acid molecule that is capable of transporting another nucleic acid molecule, e.g., into a cell. Vectors can be, for example, plasmids, cosmids, viruses, RNA vectors or linear or circular DNA or RNA molecules that may include chromosomal, non-chromosomal, semi-synthetic or synthetic nucleic acid molecules. Exemplary vectors are those capable of autonomous replication (episomal vector) or expression of nucleic acid molecules which they contain (expression vectors). In this context, the nucleic acid that is expressed is the nucleic acid encoding a peptide as described herein above. For example, expression vectors typically contain promoter and/or enhancer sequences operatively linked to the sequence to facilitate expression of the peptide with the appropriate transcription factors. Many vector platforms, such as viral vectors are known. See, e.g., Machida, C. A. (ed.), Viral Vectors for Gene Therapy: Methods and Protocols, Humana Press, Totowa, New Jersey (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno-associated virus (AAV) vector, an adenovirus vector, a retrovirus vector, or a lentivirus vector. A specific embodiment of an AAV vector includes the AAV2.5 serotype.

In a few exemplary embodiments, the nucleic acid sequence encodes a peptide with at least 80% sequence identity to at least 10, 11, 12, 13, 14, consecutive amino acids of one of SEQ ID NO:3-8 and 12, for example, at least 80%, at least 85%, at least 90%, or at least 95% sequence identity to (including 100% sequence identity to) to at least 15 or at least 20 consecutive amino acids of one of SEQ ID NO:3-8 and 12. In some embodiments, the nucleic acid encodes a peptide with at least 80%, at least 85%, at least 90%, or at least 95% sequence identity to (including 100% sequence identity to) at least 10, at least 15, or at least 20 amino acids of one of SEQ ID NO:3-8 and 12. In some embodiments, the nucleic acid encodes a peptide that comprises or consists essentially of one of SEQ ID NO:3-8 and 12.

In some embodiments, the vector is an expression vector wherein the nucleic acid is operatively linked to a control sequence capable of effecting the expression of the nucleic acid molecule in a suitable host. Such control sequences can include a promoter to effect transcription, an optional operator sequence to control such transcription, a sequence encoding suitable mRNA ribosome binding sites, and sequences which control termination of transcription and translation.

In another aspect, the disclosure provides a cell comprising the vector as described herein. The cell is capable of expressing the immunogenic peptide from the nucleic acid. For example, the nucleic acid and/or vector can be configured for expression of the immunogenic peptide from the encoding nucleic acid within the cell. A promoter operatively linked to the nucleic acid can be appropriately configured to allow binding of the cell's RNA polymerase and one or more transcription factors to permit assembly of the transcriptional complex.

As described in more detail below, endogenous antibodies that bind to the Cd loop of the Plasmodium HAP2p protein that accompany the Plasmodium spp. in a blood meal by a mosquito host can block the fusion of the male and female gametes. This blocking prevents formation of the diploid stages in the mosquito host, thereby terminating progression of the lifecycle and preventing the mosquito from transmitting the parasite to other mammalian hosts. Accordingly, in another aspect the disclosure provides a method of preventing or inhibiting transmission of a Plasmodium from an infected subject, or a subject that is susceptible to infection. The method comprises administering to the subject an effective amount of the immunogen described herein, a vector encoding the immunogenic peptide described herein, or an effective amount of the immunogenic composition described herein. In this context, the method is a prophylactic method that inhibits or prevents further transmission from the infected subject.

The subject is any mammal susceptible to Plasmodium infection, such as a human subject. The term effective amount refers to an amount that will result in production of antibodies against the immunogenic peptide (e.g., Plasmodium HAP2p or fragment or derivative thereof). In some embodiments, the method comprises administering the effective amount two or more times to the host. This provides effective booster administration(s) that can result in higher levels of anti-HAP2p antibody in the subject.

In some embodiments, the Plasmodium can infect human hosts, such as, for example, P. falciparum, P. vivax, P. ovale, P. malariae, and P. knowlesi. In some embodiments, the Plasmodium organism is P. falciparum and the susceptible host is a human.

The method of this aspect can be performed in concert with other therapeutic interventions in the treatment of the subject's infection by the Plasmodium parasite.

In another aspect, the disclosure provides an antibody or antigen binding derivative thereof that specifically binds to an immunogenic fragment of a Plasmodium HAP2 paralog (HAP2p) protein.

Antibodies and derivatives thereof, including antibody fragments, are described in more detail below. The antibody can be monoclonal or polyclonal. In some embodiments, the antibody or antigen binding derivative thereof can be chimeric or humanized.

Representative immunogenic fragments of a Plasmodium HAP2 paralog (HAP2p) protein, including fragment sequences, are described in more detail above and are expressly incorporated into this aspect of the disclosure.

In some embodiments, the HAP2p protein is a Plasmodium falciparum HAP2p protein. In some embodiments, the HAP2p fragment comprises a peptide comprising a sequence with at least 80% sequence identity to at least 10 consecutive amino acids of a sequence corresponding to SEQ ID NO:4. In some embodiments, the HAP2p fragment comprises a peptide comprising a sequence with at least 80%, at least 85%, at least 90%, or at least 95% to at least 10, at least 11, at least 12, lease 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31 consecutive amino acids, or all the amino acids of a sequence corresponding to SEQ ID NO:4. In some embodiments, the peptide comprises or consists essentially of a sequence of SEQ ID NO:4.

In another aspect, disclosure provides a method of preventing or inhibiting transmission of a Plasmodium from a susceptible host, comprising administering to the host an effective amount of the antibody or antigen binding derivative thereof as described herein. As above, in this context the method is a prophylactic method that inhibits or prevents further transmission from the infected subject.

The disclosure also encompasses formulations appropriate for methods of administration for application to in vivo therapeutic settings in subjects (e.g., mammalian subjects with or are susceptible to having malaria, i.e., a plasmodium infection). According to skill and knowledge common in the art, the disclosed immunogenic compositions, immunogenic peptides, encoding nucleic acids, and/or vectors comprising the nucleic acids, can be formulated with appropriate carriers and non-active binders, and the like, for administration to appropriate subjects in need thereof. For example, the antibody or antigen-binding derivative thereof can be included in a therapeutic composition that includes a pharmaceutically acceptable carrier.

Appropriate antibody-based or nucleic acid/vector-based formulations and modes of administration are known in the art and are encompassed by the present disclosure.

The methods of any of these treatment aspects can be performed in concert with other therapeutic interventions in the treatment of the subject's infection by the Plasmodium parasite.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

It is generally noted that the use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense, such as in the sense of “including, but not necessarily limited to,” as opposed to an exclusive or exhaustive sense. The terms “comprise” and “comprising” can encompass embodiments that are limited to the expressed elements (i.e., consist of or consist essentially of the recited elements). Words using the singular or plural number also include the plural and singular number, respectively. Additionally, the words “herein,” “above,” and “below,” and words of similar import, when used in this application, shall refer to this application as a whole and not to any particular portions of the application. Words such as “about” and “approximately” imply minor variation around the stated value, usually within a standard margin of error, such as within 10% or 5% of the stated value. In some embodiments, “about” refers to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal with suspected of a Plasmodium infection (i.e., malaria). In certain embodiments, the mammal is a human. While subjects may be human, the term also encompasses other mammals, particularly those mammals useful as laboratory models for human disease, e.g., mouse, rat, dog, non-human primate, and the like.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

As used herein, the term “nucleic acid” refers to any polymer molecule that comprises multiple nucleotide subunits (i.e., a polynucleotide). Nucleic acids encompassed by the present disclosure can include deoxyribonucleotide polymer (DNA), ribonucleotide polymer (RNA), cDNA or a synthetic nucleic acid known in the art.

Reference to sequence identity addresses the degree of similarity of two polymeric sequences, such as protein sequences or between two nucleic acid sequences. Determination of sequence identity can be readily accomplished by persons of ordinary skill in the art using accepted algorithms and/or techniques. Sequence identity is typically determined by comparing two optimally aligned polymeric sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

As used herein, the term “antibody” encompasses antibodies and antigen binding antibody fragments thereof, derived from any antibody-producing mammal (e.g., mouse, rat, rabbit, and primate including human), that specifically bind to an antigen of interest (e.g., HAP2p). Exemplary antibodies include monoclonal antibodies; polyclonal antibodies; multi-specific antibodies (e.g., bispecific antibodies); humanized antibodies; murine antibodies; chimeric, mouse-human, mouse-primate, primate-human monoclonal antibodies, etc.; and anti-idiotype antibodies. The antigen-binding molecule can be any intact antibody molecule or fragment thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V H and V L domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the HAP2p protein. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Polyclonal antibodies can be produced by immunizing an animal with optional booster immunizations and thereafter collecting anti-sera therefrom. An exemplary description of this approach is described in more detail below. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present disclosure can also be generated using various phage display methods known in the art.

Disclosed are materials, compositions, and components that can be used for, in conjunction with, and in preparation for the disclosed methods and compositions. It is understood that when combinations, subsets, interactions, groups, etc., of these materials are disclosed each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

Exemplary Technical Description

The following description is set forth so as to provide those of ordinary skill in the art with a complete disclosure and description of how to make and use the present invention, and is not intended to limit the scope of what the inventors regard as their invention nor are they intended to represent that the experiments below are all or the only experiments performed.

The following describes the surprising discovery that a paralog of HAP2 in Plasmodium species is expressed in male gametes and is critical for gamete fusion and development of deployed zygotes in the mosquito host. Antibodies that bind to the Cd loop of the paralog, referred to as HAP2p, block gamete fusion and, therefore, transmission of active infection to the mosquito host.

The membrane protein HAP2 was first identified in plants as a male gamete fertility factor. HAP2 was characterized as a sperm-specific protein required during sperm-egg fusion. HAP2 has high homology with viral Class II fusion proteins, which are integral to viral penetration of host cells. Further analysis, including crystal structure from Arabidopsis thaliana and other plant species, as well as sequence comparison, has demonstrated that HAP2 is highly conserved and is observed in a wide range a species, including protists, choanoflagellates, algae, higher plants, and metazoans. HAP2 inserts into the target gamete membrane via a highly conserved hydrophobic fusion loop.

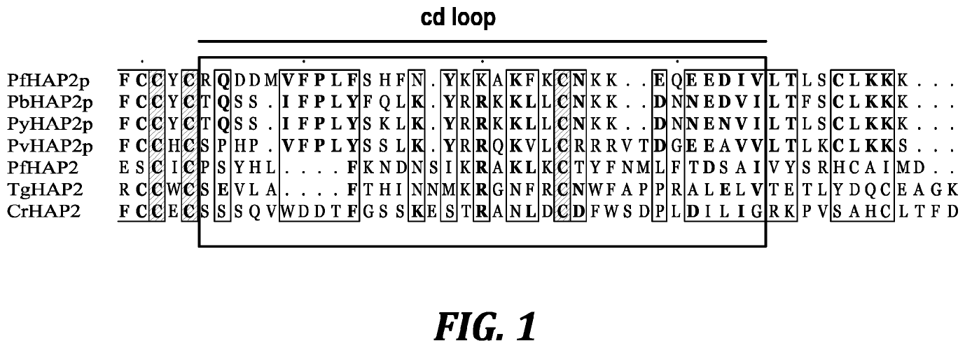

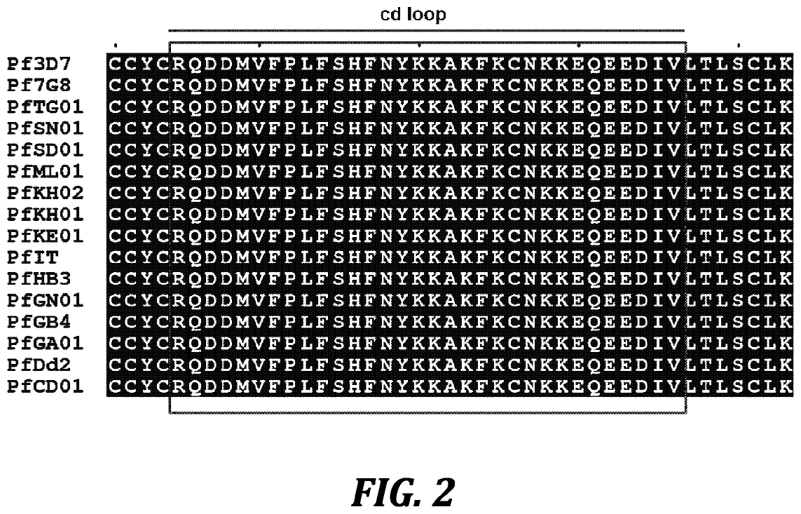

This investigation identified a Plasmodium paralog to HAP2, referred to as HAP2p, which also contains a hydrophobic loop, referred to as a Cd loop. An exemplary nucleic acid sequence encoding the HAP2p in one P. falciparum isolate is set forth herein as SEQ ID NO:1, and encodes the amino acid sequence set forth herein as SEQ ID NO:2. Sequence comparison of HAP2p fragments containing the hydrophobic Cd loop from various species of Plasmodium, including P. falciparum, P. vivax, P. berghei, and P. yoelii, indicates a high level of conservation. See FIG. 1, which is a sequence alignment of protein fragment sequences of the HAP2p protein from the various Plasmodium species encompassing the Cd loop domain. The alignment also includes the paralogous fragment from P. falciparum HAP2 protein. Isolates and strains within the P. falciparum species were demonstrated as having 100% sequence conservation at the protein level for the fragment encompassing the Cd loop domain. See FIG. 2.

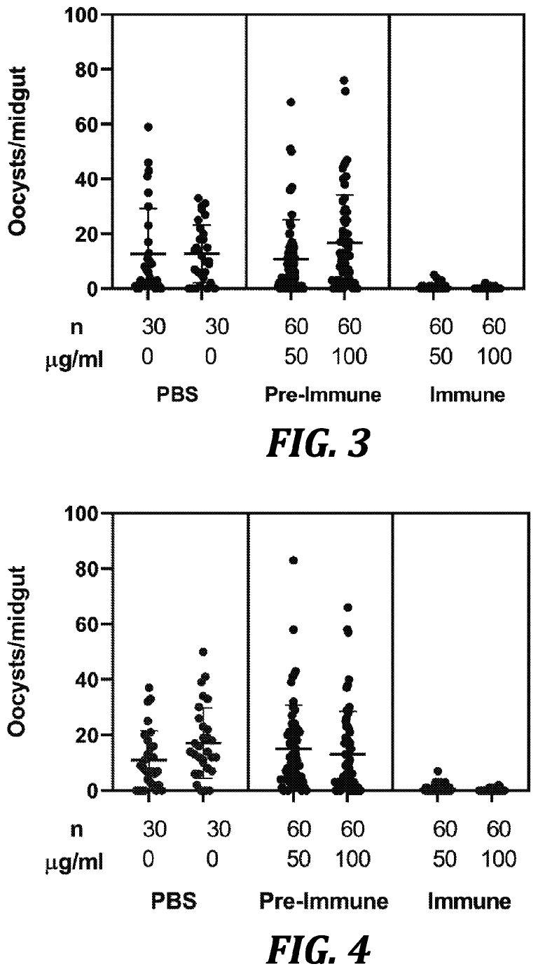

Based on the conserved Cd loop domain, a fusion immunogen was generated that fused the P. falciparum Cd loop domain sequence (SEQ ID NO:3) to keyhole limpet hemocyanin (KHL), which served as an immunogenic carrier protein. Anti-sera was collected and incorporated into initial fertilization experiments. Briefly, mosquitoes were provided blood meals containing P. falciparum gametocytes the blood meals were spiked with PBS (control), serum from pre-immunized animals, and serum from immunized animals (antibodies were included in an amount of 50 or 100 μg/ml. As illustrated in FIGS. 3 and 4, the number of resulting oocysts observed in the midgut of the mosquitos feeding mosquitos is equivalent as between the PBS control and preimmune blood meals. However, the presence of anti-Cd loop antibodies in the blood meals from immunized animals blocked fertilization and formation of oocysts in the mosquito midgut, thereby interrupting transmission from the mammalian host into the mosquito host.

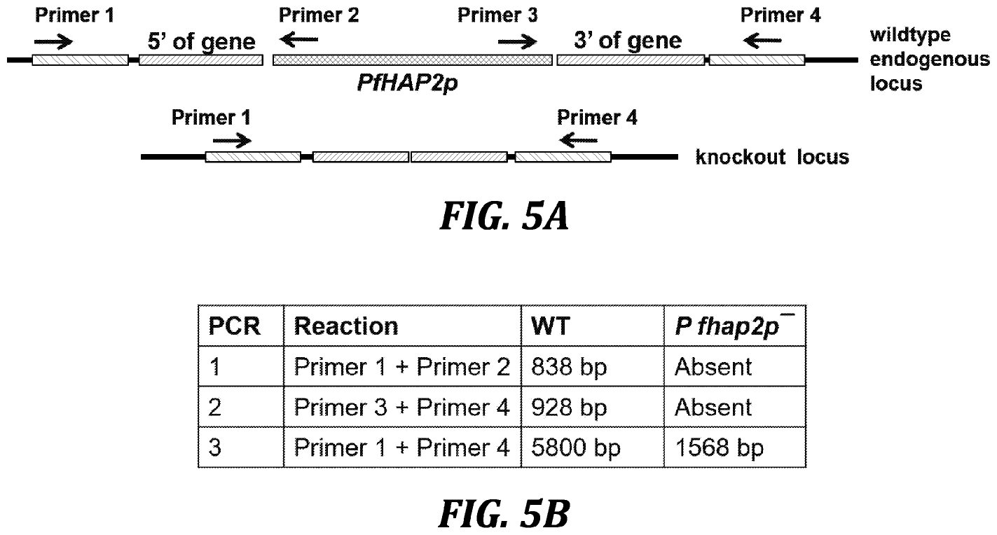

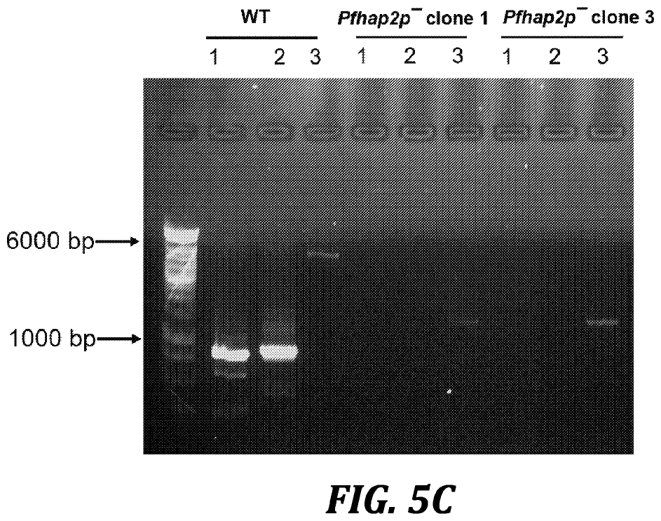

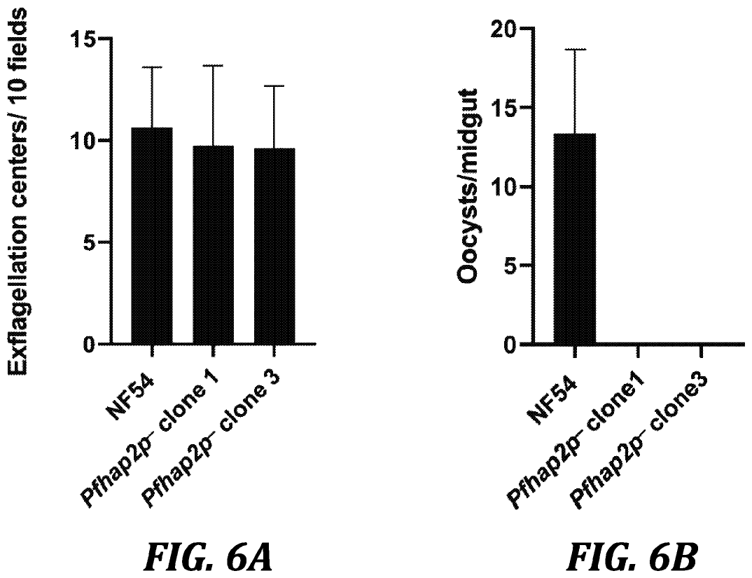

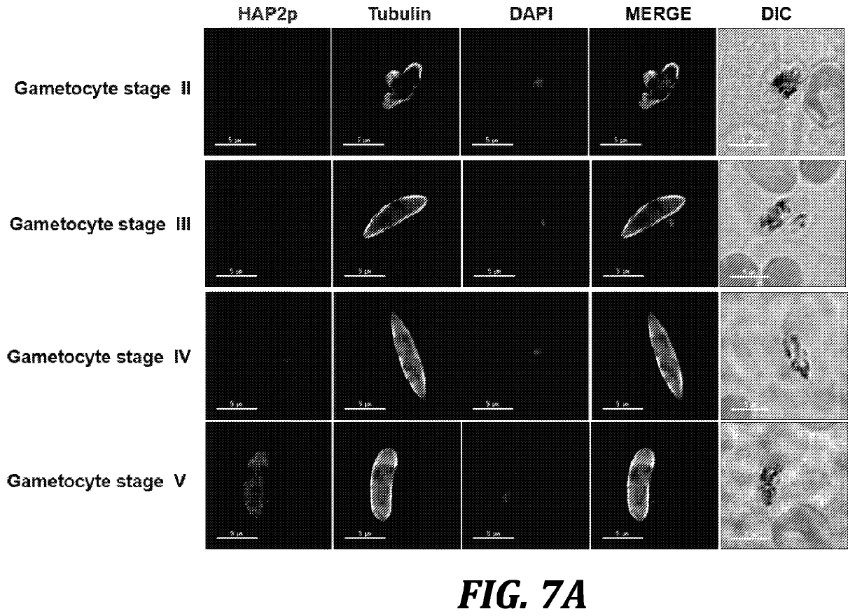

A genetic knockout strain of Plasmodium falciparum lacking the HAP2p gene (hap2p⁻) was generated to further characterize the role of this gamete fusion protein in transmission biology. Successful knockout was confirmed using PCR-based genotyping. As illustrated in FIGS. 5A-5C, primers were designed to amplify the genetic locus. Upon amplification, presence of the locus can be observed on a gel. The genetic KO P. falciparum were cultivated and fed to Anopheles mosquitoes in a blood meal. Infected mosquitoes were observed for exflagellation. FIG. 6A graphically illustrates that the genetically modified (hap2p⁻) P. falciparum underwent exflagellation at a normal rate compared to the control, i.e. NF54 strain that was not genetically modified. However, in sharp contrast to the control NF54 control parasites, the hap2p⁻ parasites did not establish oocysts in the mosquito midgut. See FIG. 6B. This demonstrates that while HAP2p is not required for normal gametocyte development and exflagellation, the hap2p⁻ gametes are not able to fuse to establish diploid zygotes or oocysts.

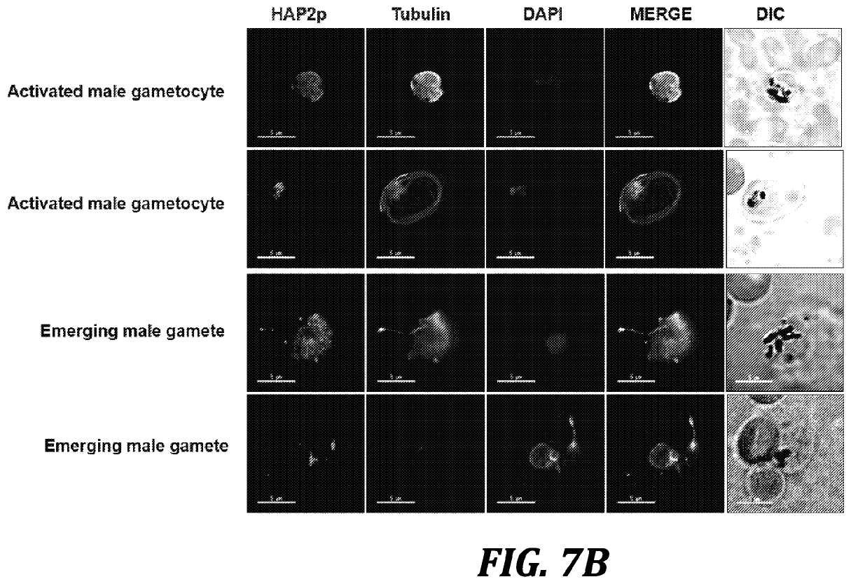

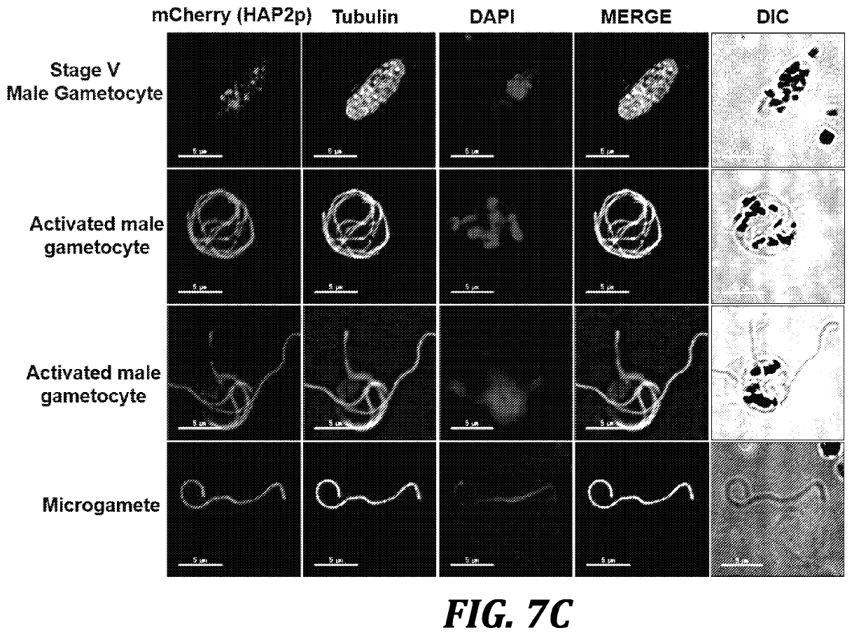

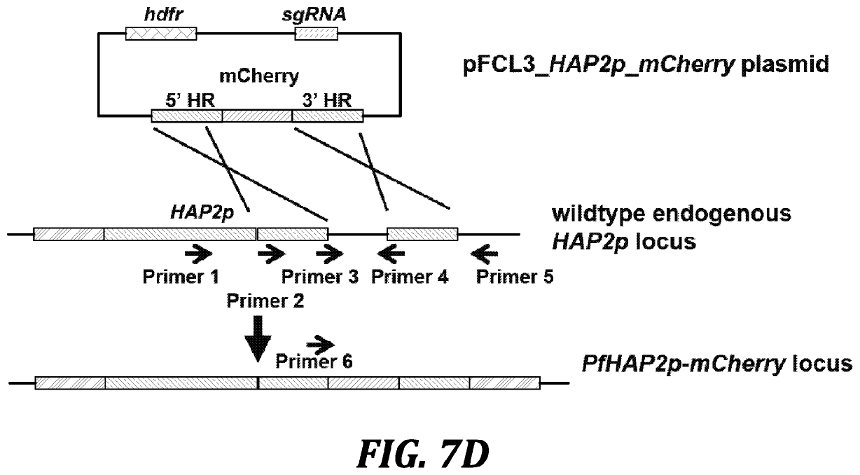

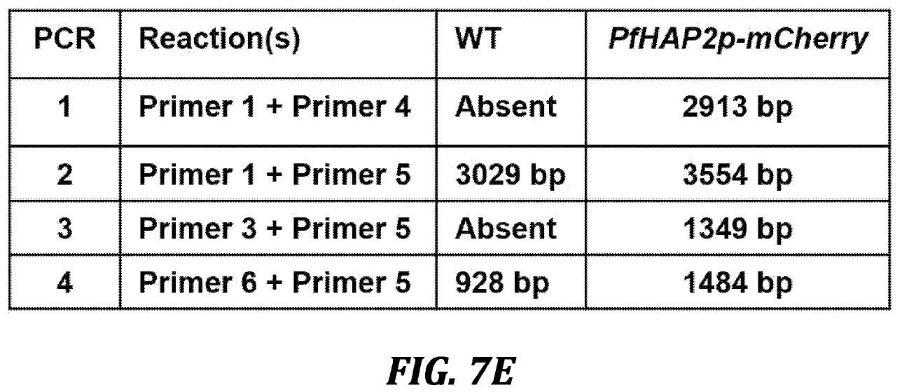

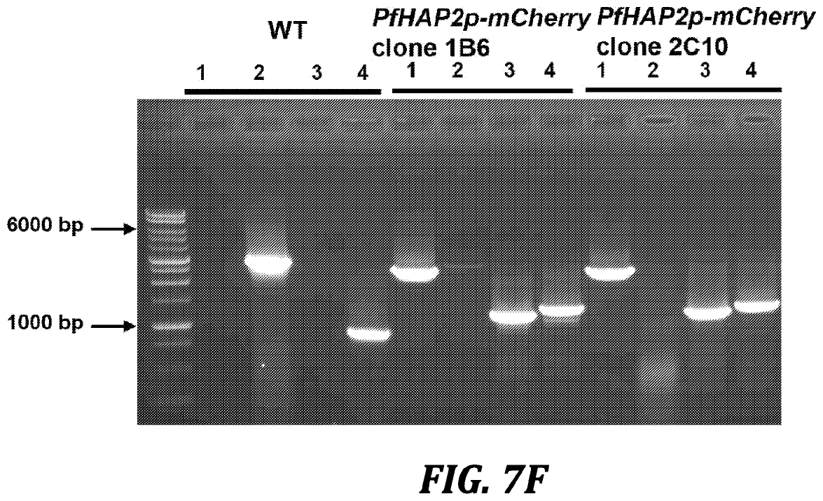

Anti-HAP2p Cd loop antibodies were incorporated into an immunofluorescence assay to characterize the expression and localization of HAP2p in developing gametocytes. As illustrated in FIGS. 7A and 7B, the HAP2p protein is first observed at significant levels in gametocyte stage V and continues through activated gametocyte stages and in emerging male gametocytes. This data demonstrates the expression of HAP2p in developed gametocytes and differentiated male gametocytes, thereby confirming its presence as a target for neutralizing or blocking antibodies, which can prevent gamete fusion and development of oocytes.

A genetically modified strain of Plasmodium falciparum with HAP2p gene tagged with fluorescent reporter mCherry (PfHAP2p-mCherry) was generated to further study the expression of this gamete fusion protein in gametocyte and gametic stages as illustrated in FIGS. 7D, 7E and 7F.

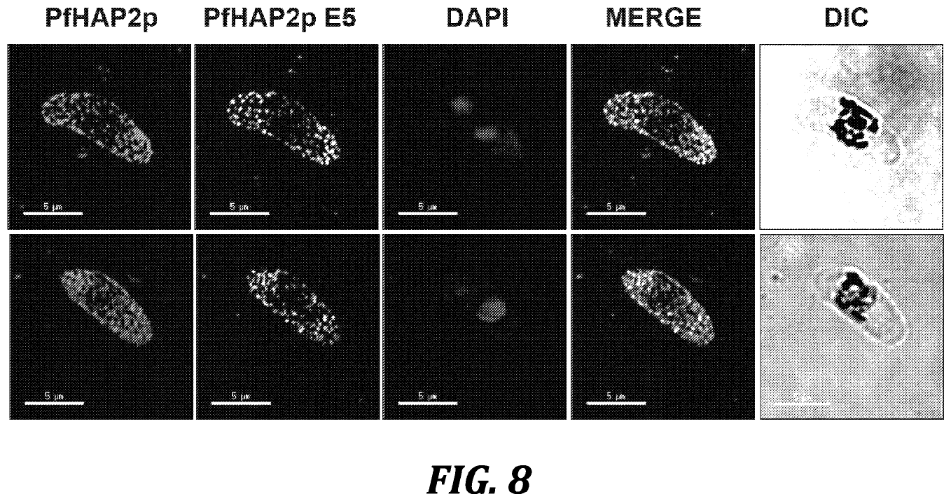

A monoclonal antibody was generated based on the conserved Cd loop domain, and a fusion immunogen was generated that fused the P. falciparum Cd loop domain sequence (SEQ ID NO:3) to keyhole limpet hemocyanin (KHL), which served as an immunogenic carrier protein. Spleen from immunized mice was collected to isolate B-lymphocyte cells for isolation of monoclonal antibodies. Antibody clone #E5 was used to test its reactivity with stage V gametocytes as illustrated in FIG. 8.

Together, these data demonstrate that the Plasmodium paralog of HAP2, referred to herein as HAP2p, is necessary for gamete fusion and development of diploid oocysts in the mosquito host. Antibodies that bind to the Cd loop of HAP2p block gamete fusion and prevent development of oocysts in the mosquito host. This effect is observed in instances when mosquitoes received the antibodies in the same blood meal as the gametocytes. This demonstrates that mammalian hosts with endogenous levels of anti-HAP2P antibodies can prevent transmission of their Plasmodium infections. Therefore, the provision of blocking antibodies and/or vaccine compositions that stimulate generation of endogenous blocking antibodies can be an effective approach to breaking the malarial lifecycle.

### INDEX OF SEQUENCES

SEQ ID NO:1—nucleic acid sequence encoding Plasmodium falciparum HAP2p; PlasmoDB ID: PF3D7_0816300, incorporated herein by reference in its entirety.

SEQ ID NO:2—amino acid sequence of Plasmodium falciparum HAP2p protein; reported in PlasmoDB ID: PF3D7_0816300, incorporated herein by reference in its entirety (Cd loop domain is from residues 95-126).

SEQ ID NO:3—amino acid sequence of a fragment of the Plasmodium falciparum HAP2p Cd loop domain fragment.

SEQ ID NO:4—amino acid sequence of the Plasmodium falciparum HAP2p Cd loop domain.

SEQ ID NO:5—amino acid sequence of a fragment of the Plasmodium falciparum HAP2p protein that contains the Cd loop domain, as illustrated in FIG. 1.

SEQ ID NO:6—amino acid sequence of a fragment of the Plasmodium berghei HAP2p protein that contains the Cd loop domain, as illustrated in FIG. 1.

SEQ ID NO:7—amino acid sequence of a fragment of the Plasmodium yoelii HAP2p protein that contains the Cd loop domain, as illustrated in FIG. 1.

SEQ ID NO:8—amino acid sequence of a fragment of the Plasmodium vivax HAP2p protein that contains the Cd loop domain, as illustrated in FIG. 1.

SEQ ID NO:9—amino acid sequence of a fragment of the Plasmodium falciparum HAP2 protein that contains the Cd loop domain, as illustrated in FIG. 1.

SEQ ID NO:10—amino acid sequence of a fragment of the Toxoplasma gondii HAP2 protein that contains the Cd loop domain, as illustrated in FIG. 1.

SEQ ID NO:11—amino acid sequence of a fragment of the Chlamydomonas reinhardtii HAP2 protein that contains the Cd loop domain, as illustrated in FIG. 1.

SEQ ID NO:12—amino acid sequence of a fragment of the Plasmodium falciparum HAP2p protein (from multiple strains) that contains the Cd loop domain, as illustrated in FIG. 2.

While illustrative embodiments have been illustrated and described, it will be appreciated that various changes can be made therein without departing from the spirit and scope of the invention.

1. An immunogen comprising:
an immunogenic fragment of a Plasmodium HAP2 paralog (HAP2p) protein; and
an immunogenic carrier operatively coupled to the peptide.

2. The immunogen of claim 1, wherein the HAP2p protein is a Plasmodium falciparum HAP2p protein.

3. An immunogen comprising:
a peptide comprising a sequence with at least 80% sequence identity to at least 10 consecutive amino acids of a sequence corresponding to SEQ ID NO:4; and
an immunogenic carrier operatively coupled to the peptide.

4. The immunogen of claim 3, wherein the peptide comprises a sequence with at least 85%, at least 90%, or at least 95% to at least 10 consecutive amino acids of a sequence corresponding to SEQ ID NO:4.

5. The immunogen of claim 3, wherein the peptide comprises a sequence with at least 80%, at least 85%, at least 90%, or at least 95% to at least 15 or at least 20 consecutive amino acids of a sequence corresponding to amino acids 61-116 of SEQ ID NO:2.

6. The immunogen of claim 3, wherein the peptide comprises a sequence with at least 80% sequence identity to at least 10 consecutive amino acids of a sequence corresponding to SEQ ID NO:3.

7. The immunogen of claim 6, wherein the peptide comprises a sequence with at least 85%, at least 90%, or at least 95% to at least 10 consecutive amino acids of a sequence corresponding to SEQ ID NO:3.

8. The immunogen of claim 6, wherein the peptide comprises a sequence with at least 80%, at least 85%, at least 90%, or at least 95% to at least 15 or at least consecutive amino acids of a sequence corresponding to SEQ ID NO:4.

9. The immunogen of claim 6, wherein the peptide comprises or consists essentially of a sequence corresponding to SEQ ID NO:4.

10. The immunogen of claim 1, wherein the immunogenic carrier is or comprises keyhole limpet hemocyanin, Concholepas concholepas hemocyanin, bovine serum albumin (BSA), cationized BSA, ovalbumin, and the like.

11. An immunogenic composition comprising the immunogen of claim 1, and an adjuvant.

12. (canceled)

13. A vector comprising a nucleic acid sequence encoding the peptide as recited in claim 3.

14-18. (canceled)

19. A method of preventing or inhibiting transmission of a Plasmodium from an infected or susceptible host, comprising administering to the host an effective amount of the immunogen as recited in claim 1.

20-21. (canceled)

22. An antibody or antigen binding derivative thereof that specifically binds to an immunogenic fragment of a Plasmodium HAP2 paralog (HAP2p) protein.

23. The antibody or antigen binding derivative thereof of claim 22, wherein the HAP2p protein is a Plasmodium falciparum HAP2p protein.

24. The antibody or antigen binding derivative thereof of claim 22, wherein the HAP2p fragment comprises a peptide comprising a sequence with at least 80% sequence identity to at least 10 consecutive amino acids of a sequence corresponding to SEQ ID NO:4.

25. The antibody or antigen binding derivative thereof of claim 24, wherein the HAP2p fragment comprises a peptide comprising a sequence with at least 80%, at least 85%, at least 90%, or at least 95% to at least 15 or at least 20 consecutive amino acids of a sequence corresponding to SEQ ID NO:4.

26. The antibody or antigen binding derivative thereof of claim 22, wherein the HAP2p fragment comprises a peptide comprising a sequence with at least 80%, at least 85%, at least 90%, or at least 95% to at least 10, at least 15, or at least 20 sequence identity of a sequence corresponding to SEQ ID NO:3.

27. The antibody or antigen binding derivative thereof of claim 26, wherein the peptide comprises or consists essentially of a sequence corresponding to SEQ ID NO:3.

28. (canceled)

29. A method of preventing or inhibiting transmission of a Plasmodium from susceptible host, comprising administering to the host an effective amount of the antibody or antigen binding derivative thereof as recited in claim 22.
